(19) United States
(12) Patent Application Publication
(10) Pub. No.: US 2025/0042846 A1
(43) Pub. Date: Feb. 6, 2025

(54) METHOD FOR PREPARING A SULFUR OR SELENISED COMPOUND BY THIOLENE REACTION

(71) Applicant: ADISSEO FRANCE S.A.S., Antony (FR)

(72) Inventors: Virginie BELLIERE-BACA, Millery (FR); Sébastien PERDRIAU, Le Creusot (FR); Mylène ROUDIER, Chassieu (FR); Jérôme MONBRUN, Dax (FR)

(21) Appl. No.: 17/600,460

(22) PCT Filed: Apr. 2, 2020
(86) PCT No.: PCT/EP2020/059380

§ 371 Date: Sep. 30, 2021

(30) Foreign Application Priority Data
Apr. 4, 2019 (FR) FR1903619

(51) Int. Cl. C07C 319/18

(52) U.S. Cl. CPC C07C 319/18

(57) ABSTRACT

The invention relates to a method for preparing a compound of formula (I)

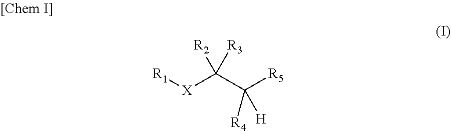

wherein
X is selected from S, Se;
R₁ is selected from the alkyl, aryl, alkylaryl and heteroaryl groups, possibly carrying one or several function(s),
R₂, R₃ and R₄ are selected, independently of each other, amongst H and the alkyl, aryl and alkylaryl groups; or R₂ and R₄ form together a C5-C10 carbon cycle;
R₅ is selected from H and the alkyl, aryl and alkylaryl groups, or represents a hydrocarbon group or chain, possibly functionalized,
which method comprises the reaction of a compound of formula (IV)

[Chem IV]

R₁—(X)ₘ—R₂₁ (IV)
wherein
X is selected from S and Se;
m=1 or 2;
R₁ is as defined hereinabove for the compound (I); and
if m=1, R₂₁ represents H;
if m=2, R₂₁ is selected from the alkyl, aryl and alkylaryl groups.
with a compound of formula (V):

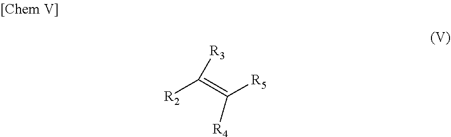

where R₂, R₃, R₄ and R₅ are as defined hereinabove for the compound of formula (I),
by irradiation with radiations having a wavelength from 200 to 800 nm in the presence of at least one compound carrying at least one function selected from the alcohol, carboxylic acid, thioether and selenoether functions.

### TECHNICAL FIELD

The disclosure concerns a method for preparing a sulfur or selenised compound of formula (I)

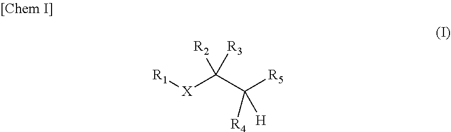

wherein
X is selected from S, Se;
R₁ is selected from the alkyl, aryl, alkylaryl and heteroaryl groups, possibly carrying one or several function(s), said functions being selected from
the hydroxyl function and the derivative functions thereof like the ether function,
the carbonyl function like the ketone and aldehyde functions, and the derivative functions thereof like the hemiacetal and acetal functions, and
the carboxylic acid function and the derivative functions thereof like the carboxylic acid ester functions;
R₂, R₃ and R₄ are selected, independently of each other, amongst H and the alkyl, aryl and alkylaryl groups; or R₂ and R₄ form together a C5-C10 carbon cycle;
R₅ is selected from H and the alkyl, aryl and alkylaryl groups,
or R₅ meets formula (II)

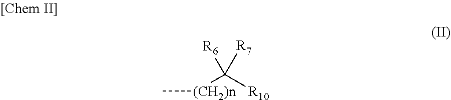

where
n=0-24
R₆ and R₇ are selected, independently of each other, from H, the alkyl, aryl and alkylaryl groups, OR₈, NR₈R₉ where R₈ and R₉ are selected, independently of each other, from H and the alkyl, aryl, alkylaryl and acyl groups;
R₁₀ is selected from H, the alkyl, aryl and alkylaryl groups, OR₁₁ and NR₁₁R₁₂ where R₁₁ and R₁₂ are selected, independently of each other, from H, the alkyl, aryl, alkylaryl and alkylaryl groups,
or R₁₀ is selected from CN, COR₁₃, COOR₁₃ and CONR₁₃R₁₄, where R₁₃ and R₁₄ are selected, independently of each other, from H and the alkyl, aryl and alkylaryl groups,
or R₁₀ represents CH₂OR₁₅ where R₁₅ is selected from H and the alkyl, aryl, alkylaryl and acyl groups,
or R₅ meets formula (III)

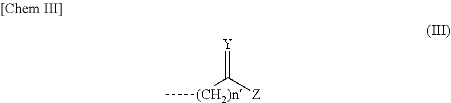

where
n′=0-24
Y is selected from O and NR₁₆ where R₁₆ is selected from the alkyl, aryl, alkylaryl groups, OR₁₄ where R₁₄ is as defined before
Z is selected from H, the alkyl, aryl, alkylaryl groups, CN,
COR₁₇ where R₁₇ is selected from H, the alkyl, aryl, alkylaryl groups, from OR₁₈, NR₁₈R₁₉ where R₁₈, R₁₉ and R₂₀ are selected, independently of each other, from H and the alkyl, aryl and arylalkyl groups, and CH₂OR₂₀ where R₂₀ is selected from H and the alkyl, aryl, alkylaryl and acyl groups, and
OR₁₈ and NR₁₈R₁₉ where R₁₈ and R₁₉ are selected, independently of each other, from H and the alkyl, aryl and arylalkyl groups.

Major examples representing these compounds include 2-hydroxy-4-methylthio-butyric acid (HMTBA or MHA) and analogues thereof such as salts thereof, chelates thereof, in particular metallic chelates (of Zn, Ca, Mn, Mg, Cu, Na . . . ), esters thereof, like isopropylic and tertiobutylic esters of HMTBA, which are widely used in animal nutrition. The selenised derivatives of these hydroxy analogues of methionine are also major interesting constituents in animal nutrition.

### BACKGROUND

The preparation of HMTBA is well known and could be done by different processes involving various synthesis intermediates, and in particular acrolein and methanethiol. 2-hydroxy-4-methylseleno-butyric acid is also easily accessible by similar synthesis processes. The drawback of processes implemented on an industrial scale lies in the production of considerable volumes of salts resulting from the neutralization allowing accessing the end product, such as ammonium sulfate or sodium sulfate, the separation of which requires heavy purification treatments involving large amounts of solvent, and the recycling of which is still difficult.

The fact that most of these processes require intermediates obtained from only propylene as acrolein source, an essential intermediate, is another major drawback.

### SUMMARY

The present disclosure provides an alternative to existing processes and further allows access for a large number of sulfur or selenised compounds, the applications of which are, of course, not limited to animal nutrition. Thus, such compounds could be used in many fields, namely in the composition and/or the preparation of surfactants, monomers, polymers, plasticizers, adhesives, coatings, lacquers, films, emulsifiers, antioxidants, antimicrobial agents, anticorrosive agents, packaging materials, consumer products, as well as in medical or agricultural applications.

According to the method of the disclosure, a compound of formula I hereinabove comprises the reaction of a compound of formula (IV)

[Chem IV]

R₁—(X)ₘ—R₂₁ (IV)
wherein
X is selected from S and Se;
m=1 or 2;
R₁ is as defined hereinabove for the compound (I), that is to say it is selected from the alkyl, aryl, alkylaryl and heteroaryl groups, possibly carrying one or several function(s), said functions being selected from
the hydroxyl function and the derivative functions thereof like the ether function,
the carbonyl function like the ketone and aldehyde functions, and the derivative functions thereof like the hemiacetal and acetal functions, and
the carboxylic acid function and the derivative functions thereof like the carboxylic acid ester functions; and
if m=1, R₂₁ represents H;
if m=2, R₂₁ is selected from the alkyl, aryl and alkylaryl groups.
with a compound of formula (V):

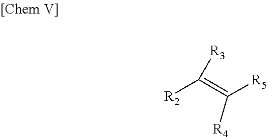

where R₂, R₃, R₄ and R₅ are as defined hereinabove for the compound of formula (I),
by irradiation with radiations having a wavelength from 200 to 800 nm in the presence of at least one compound carrying at least one function selected from the alcohol, carboxylic acid, thioether and selenoether functions, said compound being represented by formula (VI).

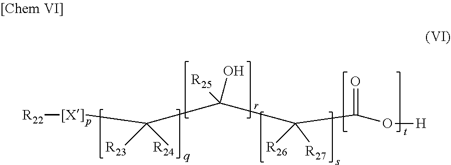

wherein
X′ is selected from S or Se;
p and t amount, independently of each other, to 0 or 1;
q, r and s amount, independently of each other, to 0 to 10;
provided that p+r+t is greater than or equal to 1 and if p≠0 while q+s≠0;
R₂₂ is selected, if p=0, from H and the alkyl, aryl and arylalkyl groups and, if p=1, from the alkyl, aryl and arylalkyl groups; and
R₂₃, R₂₄, R₂₅, R₂₆ and R₂₇ are selected, independently of each other, from H and the alkyl, aryl and alkylaryl groups.

The features, applications and advantages of the disclosure are disclosed hereinafter in more details, bearing in mind that these features could be considered independently of each other, or in combination, regardless of the combination.

Before this description, some used terms are defined hereinafter.

In the formulas defining the obtained or involved compounds, by the term «alkyl», it should be understood a linear or branched, saturated hydrocarbon monovalent radical, having from 1 to 20 carbon atoms, advantageously from 1 to 6 carbon atoms, such as methyl, ethyl, propyl, isopropyl, butyl, isobutyl, tert-butyl, pentyl, neopentyl, n-hexyl, or a cyclic, saturated hydrocarbon monovalent radical, having from 3 to 20 carbon atoms, advantageously from 5 to 7 carbon atoms, such as cyclopentyl, cycloheptyl.

By «aryl» group, it should be understood an aromatic hydrocarbon monovalent radical comprising from 6 to 22 carbon atoms which could be functionalized by methoxy or ester groups for example, as illustrated by the phenyl, naphtyl, anisole, alkylbenzoate groups where the term alkyl has the definition provided before.

By «alkylaryl» group, it should be understood an aryl group comprising from 6 to 22 carbon atoms, said aryl group being substituted by at least one alkyl group complying with the definition hereinabove, as illustrated by the tolyl, mesityl, xylyl groups; this term indifferently refers to an alkyl group complying with the definition hereinabove that is substituted with at least one aryl group complying with the definition hereinabove as illustrated by the benzyl, benzyhydryl, phenethyl, trityl groups.

The term «heteroaryl» defines an aromatic hydrocarbon monovalent radical comprising from 3 to 21 carbon atoms and at least one heteroatom such as O, N, as illustrated by the pyrrolyl, pyridyl, indolyl, furyl groups.

The term «acyl» defines a monovalent radical RC(O)— wherein R is an alkyl group as specified hereinabove.

### DETAILED DESCRIPTION OF THE DISCLOSURE

The compound of formula (VI) according to the disclosure is also called additive. In the formula (VI) as defined hereinabove, and from the condition according to which p+r+t should be greater than or equal to 1, it comes out that an additive according to the disclosure is a hydrocarbon compound which includes at least one function selected from the thioether, selenoether, primary alcohol/hydroxyl, secondary alcohol/hydroxyl, tertiary alcohol/hydroxyl and carboxylic acid functions. It could comprise at least two or at least three of said functions, or more, said functions being identical or different. According to the disclosure, the method may be implemented in the presence of a mixture of different compounds of formula (VI); in this variant, each of the compounds of formula (VI) may bring in one or more of said functions. The formula (VI) should be understood as covering the compounds for which, when q, r and/or s are greater than 1, while the substituents R₂₃, R₂₄, R₂₅, R₂₆ and R₂₇ may represent, in a generic manner, respectively different substituents R₂₃′, R₂₃″, R₂₃′″ . . . , R₂₄′, R₂₄″, R₂₄′″ . . . , R₂₅′, R₂₅″, R₂₅′″ . . . , R₂₆′, R₂₆″, R₂₆′″ . . . , and R₂₇′, R₂₇″, R₂₇′″ . . . ; as example, a compound (VI) may consist of 4-methyl-1,2-butanediol.

The method of the disclosure should be conducted in the presence of an irradiation with radiations having a wavelength from 200 to 800 nm. This feature is essential. By comparing an irradiation with a wave radiation in the visible light spectrum and an irradiation with an UV radiation, with identical involved compounds, although the reaction takes place in both situations, it is observed that its yield is significantly higher when the radiation is UV. Thus, the method of the disclosure is advantageously operated under a radiation at a wavelength from 254 to 400 nm, ideally 365 nm±20 nm. According to a preferred variant, the exposure to the radiation is continuous.

The adequate irradiation conditions as described hereinabove may be ensured by a light-emitting diode (LED) yet without being limited thereto.

A more detailed description of a compound (VI) is provided hereinafter.

As indicated hereinabove, the additive in the method according to the disclosure is a compound (VI) that comprises at least one function selected from the alcohol, carboxylic acid, thioether and selenoether functions; it may thus comprise 2 or more of said functions, these being identical or different. According to one variant, the method of the disclosure may involve several ones of said additives, these respectively carrying one or more of said functions, identical or different. As an illustration of a method involving several additives, these will comprise the mixture of a compound carrying a thioether function or a selenoether and of a compound carrying a hydroxyl and/or carboxylic acid function. According to a particular implementation that is advantageous in terms of selectivity of the reaction, the compound (VI) is an α-hydroxyacid. Indeed, it has been noticed that for a compound (VI) comprising a hydroxyl function and a carboxylic acid function, the performances increase when bringing the hydroxyl and carboxylic acid functions together. For illustration, such a hydroxyacid may consist of lactic acid, glycolic acid, 2-hydroxyisobutyric acid. Based on this definition, those skilled in the art have the general knowledge necessary to retain one or more of the compounds (VI) as additive(s) according to the disclosure.

As non-limiting examples allowing illustrating the formula (VI), the compound is selected from alkyl sulfides such as alkyl sulfides such as methyl sulfide and ethyl sulfide, propionic acid, butanoic acid, lactic acid, 3-hydroxy-proprionic acid, 3-hydroxy-butyric acid, 6-hydroxycaproic acid, 3-methylthiopropanoic acid, 4-methylthiobutanol, 2,4-hydroxybutyric (2,4-DHB) acid, 4-methylthio-2-hydroxybutyric acid (MHA) and methylthiobutanediol (MTBDO), these compounds may be used alone or in mixtures of 2, 3 or more.

The compound (VI) is present in the reaction medium in a preferred amount of at least 0.1 eq, and even better at least 0.2 eq, for 1 eq of the compound (V). Indeed, a significant gain in terms of selectivity of the reaction has been observed as soon as the compound (VI) is present in the reaction medium, in the conditions of the disclosure defined hereinabove, even in small amounts. Up to an amount of about 0.5 eq of the compound (VI), an increase in the reaction speed and in the selectivity is measured. An excess of the compound (VI), for example beyond 10 eq, and even beyond 5 eq, does not allow increasing the performances of the reaction and beyond, a decrease in the reaction yield could be noticed. According to an optimum variant, the amount of the compound (VI) is at least 0.5 eq and at most 2 eq, for 1 eq of the compound (V).

A more detailed description of a compound (IV) is provided hereinafter.

A compound (IV) may be selected from any alkylthiol or alkylselenol like methanethiol, ethanethiol, n-butanethiol, tertiobutanethiol, methaneselenol, as well as any alkylthiol or alkylselenol carrying one or several hydroxyl, carbonyl, carboxylic acid or carboxylic ester function(s), like thioglycolic acid. It may also consist of a disulfide or a diselenide, in particular any alkyl disulfide or diselenide, such as dimethyl or aryl disulfide, such as diphenyl disulfide and diphenyl diselenide.

It has been noticed that the presence, in the compound (IV) of one or several function(s) that should be present in the compound (VI), would significantly increase reactivity. This effect has been observed in particular with the thioglycolic acid.

Preferably, the compound (IV) is used in excess with respect to the compound (V). In general, the molar ratio of the compound (IV) to the compound (V) is in the range of 1.1-15:1, or from 1.2-10:1. This ratio depends essentially on the involved compounds (IV) and (V) and it falls upon those skilled in the art to determine it. For indication, if the compound (IV) is a sulfide or a selenide, the ratio of the compound (IV) to the compound (V) is rather in the range of 1.1-2:1; when the compound (IV) is a disulfide or a diselenide, the compound (IV) excess is higher and this ratio is in the range of 5-10:1.

A more detailed description of a compound (V) is provided hereinafter.

In accordance with the formula (V) defined hereinabove, the compound (V) includes a carbon-carbon double bond on which the compound (IV) will react according to the thiolene reaction. This reaction is known to those skilled in the art who shall be able to select the compounds (IV) and (V) respectively, according to the pursued compound (I), as well as according to the reactivity of the functions contained in the formulas of these compounds (IV) and (V) in this thiolene reaction. Hence, all compounds (IV) and (V) that could react by thiolene addition could be applied to the method of the disclosure. As non-limiting examples, a compound (V) is selected from butene, pentene, hexene, 2,3-dimethyl-butene, but-3-enoic acid, but-2-enoic acid, but-3-en-2-ol, butene-diol, cyclohexene, vinyl glycolic acid (VGA), methyl vinyl glycolate (MVG). VGA and MVG are compounds derived from biomass and therefore constitute a natural and abundant source of compound (V), conferring on the present disclosure another attractiveness for its use on an industrial scale

The method of the disclosure is also interesting in that it could be conducted at a temperature close to room temperature. Thus, it has been experimented that while a temperature in the range of 32° C.±5° C. is optimum, its increase up to 50° C. leads to a comparable kinetic profile, yet with a reduction of selectivity. Thus, the temperature range lies between −10 and 100° C., and more particularly from 0 to 50° C. and even better from 20 to 35° C.

According to a variant of the disclosure, the reaction may be conducted in the presence of at least one photoinitiator, the latter having as effect, in the context of the disclosure, the acceleration of the reaction. This will be selected from type I or II photoinitiators and more advantageously from type II photoinitiators. As example, as type I photoinitiator, mention may be made of benzoin and 2,2-dimethoxy-2-phenylacetophenone (DMPA) and, as type II photoinitiator, mention may be made of thioxanthone and derivatives thereof like 1-chloro-4-hydroxy-thioxanthone, 1-chloro-4-propoxy-thioxanthone, or benzophenone and derivatives thereof, in particular those selected from 3-alkyl-benzophenone and 4-alkyl-benzophenone, for example 3-methylbenzophenone. When one or several photoinitiator(s) is/are used, the/their amount(s) is/are that/those conventionally used and falling without the general knowledge of those skilled in the art.

The disclosure is also interesting in that it could be carried out in the absence of any solvent. That being so, the present disclosure covers any method wherein a solvent would be used, for example to dilute the compound (VI) and in this case the solvent could be polar, protic or aprotic, and in particular selected amongst methanol and acetonitrile.

### EXAMPLES

The disclosure and its advantages are illustrated in the following examples.

In these examples, the performances of the disclosure are assessed through the determination of the following parameters:
the conversion, that is to say the transformation ratio of the compound (V) expressed in %,
the yield which defines the ratio in % of the formed compound (I) to the compound V that is brought in, and
the selectivity which defines the ratio in % of the formed compound (I) to the compound V that is actually converted,
for the preparation of compounds of formula (I) through the reaction of a compound of formula (IV) and of a compound of formula (V) under irradiation in the presence of an additive of formula (VI) or in the absence of such an additive.

### Example 1: Synthesis of (n-butylthio)-cyclohexane in the Presence of Different Additives According to the Disclosure and Comparison with the Synthesis of the Same Compound without any Additive

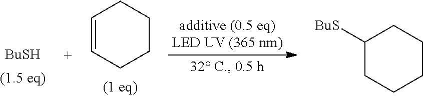

In a 20 mL mini-reactor provided with a magnetic bar, cyclohexene (500 mg), butanethiol (1.5 eq) and the additive (0.5 eq), where indicated, are successively introduced. The mixture is irradiated with a LED (365 nm) at 32° C. for 30 minutes. The performances of the reactions are calculated by 1H NMR analysis (vs 3,5-dimethylanisol used as an internal control and counted at 99%) and are illustrated in Table 1 hereinbelow.

TABLE 1

| | Additive | Conversion | Yield | Selectivity |
|---|---|---|---|---|
| No. | (0.5 eq) | (%) | (%) | (%) |
| 1 | without | 85 | 76 | 90 |
| 2 | MeSEt | 96 | 95 | 99 |
| 3 | lactic acid | 97 | 96 | 99 |
| 4 | 1,2-butanediol | 100 | 98 | 98 |
| 5 | MHA | 99 | 98 | 99 |

It appears that all tested additives unexpectedly act quite positively on all of the measured parameters.

### Example 2: Synthesis of (n-butylthio)-2-butanol in the Presence of Different Additives According to the Disclosure and Comparison with the Synthesis of the Same Compound without any Additive

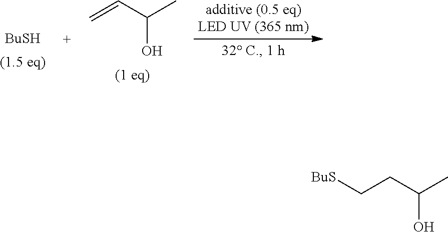

In a 20 mL mini-reactor provided with a magnetic bar, but-3-en-2-ol (500 mg), butanethiol (1.5 eq) and the additive (0.5 eq), where indicated, are successively introduced. The mixture is irradiated with a LED (365 nm) at 32° C. for 1 hour. The performances of the reactions are calculated by 1H NMR analysis (vs 3,5-dimethylanisol used as an internal control and counted at 99%) and are illustrated in Table 2 hereinbelow.

TABLE 2

| | Additive | Conversion | Yield | Selectivity |
|---|---|---|---|---|
| No. | (0.5 eq) | (%) | (%) | (%) |
| 1 | without | 42 | 31 | 73 |
| 2 | lactic acid | 99 | 98 | 99 |
| 3 | Lactic acid + MeSEt | 90 | 88 | 98 |
| | (1:1) | | | |
| 4 | MHA | 100 | 97 | 97 |

It appears that all tested additives unexpectedly act quite positively on all of the measured parameters. This example further shows that the preparation of the compound (I) could be performed in the presence of several additives functionalized in different manners.

### Example 3: Synthesis of (n-butylthio)-2-hydroxy-butanoic Acid in the Presence of 4-methyl-thio-2-hydroxy-butanoic Acid (MHA) as an Additive According to the Disclosure and Comparison with the Synthesis of the Same Compound without any Additive

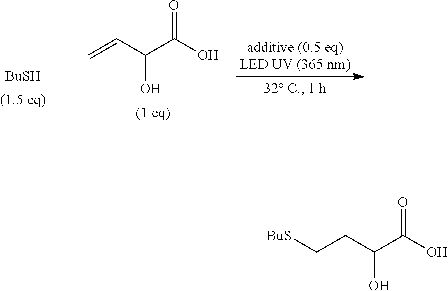

In a 20 mL mini-reactor provided with a magnetic bar, 2-hydroxy-3-butenoic acid (500 mg), butanethiol (1.5 eq) and the additive (0.5 eq), where indicated, are successively introduced. The mixture is irradiated with a LED (365 nm) at 32° C. for 1 hour. The performances of the reactions are calculated by 1H NMR analysis (vs 3,5-dimethylanisol used as an internal control and counted at 99%) and are illustrated in Table 3 hereinbelow.

TABLE 3

| | Additive | Conversion | Yield | Selectivity |
|---|---|---|---|---|
| No. | (0.5 eq) | (%) | (%) | (%) |
| 1 | without | 63 | 51 | 81 |
| 2 | MHA | 98 | 96 | 98 |

This example highlights the interest of MHA as an additive in the synthesis of another compound (I).

### Example 4: Synthesis of the Methyl Ester of 4-methylthio-2-hydroxy-butanoic Acid in the Presence of MHA as an Additive According to the Disclosure and Comparison with the Synthesis of the Same Compound without any Additive

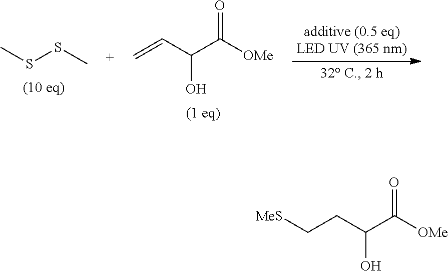

In a 20 mL mini-reactor provided with a magnetic bar, methyl 2-hydroxy-3-butenoate (500 mg), dimethylsulfide (10 eq) and the additive (0.5 eq), where indicated, are successively introduced. The mixture is irradiated with a LED (365 nm) at 32° C. for 2 hours. The performances of the reactions are calculated by 1H NMR analysis (vs 3,5-dimethylanisol used as an internal control and counted at 99%) and are illustrated in Table 5 hereinbelow.

TABLE 4

| | Additive | Conversion | Yield | Selectivity |
|---|---|---|---|---|
| No. | (0.5 eq) | (%) | (%) | (%) |
| 1 | without | 68 | 31 | 45 |
| 2 | MHA | 72 | 38 | 52 |

Even though the degree is lesser in comparison with the previous examples, a positive influence of an additive of formula (V) according to the disclosure is observed.

1. A method for preparing a compound of formula (I) 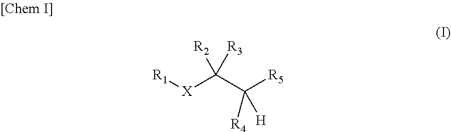
wherein
X is selected from S, Se;
R₁ is selected from the alkyl, aryl, alkylaryl and heteroaryl groups, possibly carrying one or several function(s), said functions being selected from
the hydroxyl function and the derivative functions thereof like the ether function,
the carbonyl function like the ketone and aldehyde functions, and the derivative functions thereof like the hemiacetal and acetal functions, and
the carboxylic acid function and the derivative functions thereof like the carboxylic acid ester functions;
R₂, R₃ and R₄ are selected, independently of each other, amongst H and the alkyl, aryl and alkylaryl groups; or R₂ and R₄ form together a C5-C10 carbon cycle;
R₅ is selected from H and the alkyl, aryl and alkylaryl groups,
or R₅ meets formula (II) 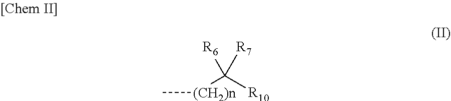
where
n=0-24
R₆ and R₇ are selected, independently of each other, from H, the alkyl, aryl and alkylaryl groups, OR₈, NR₈R₉ where R₈ and R₉ are selected, independently of each other, from H and the alkyl, aryl, alkylaryl and acyl groups;
R₁₀ is selected from H, the alkyl, aryl and alkylaryl groups, OR₁₁ and NR₁₁R₁₂ where R₁₁ and R₁₂ are selected, independently of each other, from H, the alkyl, aryl, alkylaryl and acyl groups,
or R₁₀ is selected from CN, COR₁₃, COOR₁₃ and CONR₁₃R₁₄, where R₁₃ and R₁₄ are selected, independently of each other, from H and the alkyl, aryl and alkylaryl groups,
or R₁₀ represents CH₂OR₁₅ where R₁₅ is selected from H and the alkyl, aryl, alkylaryl and acyl groups,
or R₅ meets formula (III) 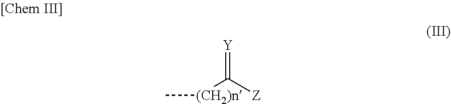
where
n′=0-24
Y is selected from O and NR₁₆ where R₁₆ is selected from the alkyl, aryl, alkylaryl groups, OR₁₄ where R₁₄ is as defined before
Z is selected from H, the alkyl, aryl, alkylaryl groups, CN,
COR₁₇ where R₁₇ is selected from H, the alkyl, aryl and alkylaryl groups and from the OR₁₈, NR₁₈R₁₉ groups where R₁₈, R₁₉ and R₂₀ are selected, independently of each other, from H and the alkyl, aryl and arylalkyl groups, and CH₂OR₂₀ where R₂₀ is selected from H and the alkyl, aryl, alkylaryl and acyl groups, and
OR₁₈ and NR₁₈R₁₉ where R₁₈ and R₁₉ are selected, independently of each other, from H and the alkyl, aryl and arylalkyl groups,
which method comprises the reaction of a compound of formula (IV)
[Chem IV]
R₁—(X)ₘ—R₂₁ (IV)
wherein
X is selected from S and Se;
m=1 or 2;
R₁ is as defined hereinabove for the compound (I), that is to say it is selected from the alkyl, aryl, alkylaryl and heteroaryl groups, possibly carrying one or several function(s), said functions being selected from
the hydroxyl function and the derivative functions thereof like the ether function,
the carbonyl function like the ketone and aldehyde functions, and the derivative functions thereof like the hemiacetal and acetal functions, and
the carboxylic acid function and the derivative functions thereof like the carboxylic acid ester functions; and
if m=1, R₂₁ represents H;
if m=2, R₂₁ is selected from the alkyl, aryl and alkylaryl groups.
with a compound of formula (V): 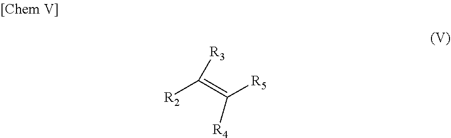
where R₂, R₃, R₄ and R₅ are as defined hereinabove for the compound of formula (I),
by irradiation with radiations having a wavelength from 200 to 800 nm in the presence of at least one compound carrying at least one function selected from the alcohol, carboxylic acid, thioether and selenoether functions, said compound being represented by formula (VI), 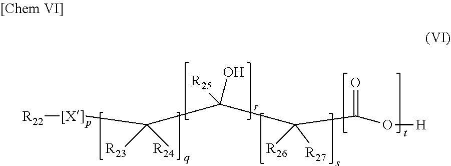
wherein
X′ is selected from S or Se;
p and t amount, independently of each other, to 0 or 1;
q, r and s amount, independently of each other, to 0 to 10;
provided that p+r+t is greater than or equal to 1 and if p≠0 while q+s≠0;
R₂₂ is selected, if p=0, from H and the alkyl, aryl and arylalkyl groups and, if p=1, from the alkyl, aryl and arylalkyl groups; and
R₂₃, R₂₄, R₂₅, R₂₆ and R₂₇ are selected, independently of each other, from H and the alkyl, aryl and alkylaryl groups.

2. The method according to claim 1, characterized in that the radiation has a wavelength from 254 to 400 nm, preferably 365 nm±20 nm.

3. The method according to claim 1 or 2, characterized in that the compound (VI) comprises at least alkyl sulfides such as methyl sulfide and ethyl sulfide, propionic acid, butanoic acid, lactic acid, 3-hydroxy-proprionic acid, 3-hydroxy-butyric acid, 1-hydroxycaproic acid, 3-methylthiopropanoic acid, 4-methylthiobutanol, 2,4-hydroxybutyric (2,4-DHB) acid, methylthiobutyric acid (MHA) and methylthiobutanediol (MTBDO).

4. The method according to any one of the preceding claims, characterized in that the compound (VI) has an amount of at least 0.1 eq, preferably at least 0.5 eq, and preferably at most 10 eq, still at most 5 eq, and even better at most 2 eq, for 1 eq of compound (V).

5. The method according to any one of the preceding claims, characterized in that a compound (IV) is reacted, selected from any alkylthiol or alkylselenol like methanethiol, ethanethiol, n-butanethiol, tertiobutanethiol, methaneselenol, from any alkylthiol or alkylselenol carrying one or several hydroxyl, carbonyl, carboxylic acid or carboxylic ester function(s), like thioglycolic acid, or from disulfides and diselenides, like alkyl disulfide or diselenide, such as dimethyl or aryl disulfide, such as diphenyl disulfide and diphenyl diselenide.

6. The method according to any one of the preceding claims, characterized in that a compound (V) is reacted, selected from butene, pentene, hexene, 2,3-dimethylbutene, but-3-enoic acid, but-2-enoic acid, but-3-en-2-ol, butene-diol, cyclohexene, vinyl glycolic acid (VGA) and methyl vinyl glycolate (MVG).

7. The method according to claim 6, characterized in that the compound (I) is selected from the 4-methylthio-2-hydroxybutyric acid, the 4-methylseleno-2-hydroxybutyric acid and esters thereof.

8. The method according to any one of the preceding claims, characterized in that the molar ratio of the compound (IV) to the compound (V) ranges from 1.1-15:1, still from 1.2-10:1.

9. The method according to any one of the preceding claims, characterized in that the reaction is performed in the presence of a type I or II, preferably type II, photoinitiator such as thioxanthone and derivatives thereof or benzophenone and derivatives thereof.
